(19) United States
(12) Patent Application Publication
(10) Pub. No.: US 2010/0062997 A1
(43) Pub. Date: Mar. 11, 2010

(54) USE OF IDRAPARINUX FOR EXTENDED PROPHYLAXIS OF THROMBOEMBOLISM

(75) Inventor: Gerard PILLION, Paris (FR)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS, Paris (FR)

(21) Appl. No.: 12/206,095

(22) Filed: Sep. 8, 2008

(51) Int. Cl. A61K 31/715

(52) U.S. Cl. 514/54

(57) ABSTRACT

The invention relates to the use of idraparinux for the extended prophylaxis of thromboembolism, in particular for the prevention of the recurrence of thromboembolism during 6 months after permanent discontinuation of the treatment with this drug.

### BACKGROUND OF THE INVENTION

The invention relates to the use of idraparinux for the prevention of recurrent thromboembolism for an additional 6-month period after permanent interruption of the anticoagulant treatment.

Patients who have had venous thromboembolism (hereafter “VTE”) have a risk of recurrence of approximately 30% during the first eight years after the initial episode (P. Prandoni et al., Ann. Intern. Med., 1996, 125, 1-7; The PREPIC study group, Circulation, 2005, 112, 416-422; G. Agnelli et al., Ann. Intern. Med., 2003, 139, 19-25). Most of these events occur during the first six to twelve months after the first event.

Vitamin K antagonists (hereafter “VKA”) are highly effective in reducing the risk of VTE recurrence (G. Agnelli et al., N. Engl. J. Med., 2001, 345, 169-9; S. Schulman et al., N. Engl. J. Med., 1995, 332, 1661-5), but their extended use for prophylaxis against VTE is often constrained by risk-benefit limitations and inconvenience. Indeed, because of numerous food and drug interactions and a narrow therapeutic margin their use requires regular laboratory monitoring and dose-adjustments to reduce the risk of thrombotic recurrence (underanticoagulation) or of bleeding (overanticoagulation) (J. Ansell et al., Chest, 2008, 133, 160S-198S). For these reasons, VKA are often discontinued before the recommended period of 6 to 12 months of prophylaxis for idiopathic thromboembolism. Because of the increasing rate over time of bleeding under VKA, especially major bleedings, in patients for whom long term treatment with anticoagulant is indicated, the benefit of extending prophylaxis for 6 to 12 months should be weighed against risk factors for bleeding of each patient (L. A. Linkins et al., Ann. Intern. Med., 2003, 139, 893-900).

Idraparinux, developed by sanofi-aventis, is a long-acting synthetic pentasaccharide (see structure below) that is an antithrombin-dependent inhibitor of activated factor Xa. Clinical experience with idraparinux suggests that a fixed dose, given subcutaneously once weekly and without laboratory monitoring, is effective for treatment of VTE and prevention of subsequent VTE recurrences (J. M. Herbert et al., Blood, 1998, 91, 4197-205; The PERSIST Investigators, J. Thromb. Haemost., 2004, 2, 47-53).

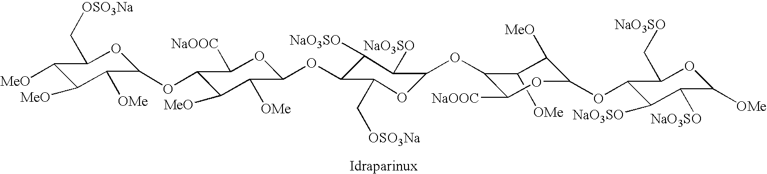

### SUMMARY OF THE INVENTION

It has now surprisingly been found that idraparinux is useful for the extended prophylaxis of thromboembolism.

### DESCRIPTION OF THE DRAWING

FIG. 1 is a graphical representation of the cumulative incidence (%) of first confirmed symptomatic recurrent venous thromboembolism versus time in days.

### DETAILED DESCRIPTION

According to the instant invention:
the term “extended” prophylaxis designates protection effective against thromboembolism in a period following discontinuation of the treatment with a given drug, i.e., a prolonged protective effect;
the term “discontinuation” designates a permanent interruption of the treatment by the patient;
the term “treatment” refers to the administration of a therapy to an individual who already manifests at least one symptom of a disease or condition—in the instant case a confirmed thromboembolic pathology such as venous thromboembolism (VTE), i.e., deep venous thrombosis (DVT) or pulmonary embolism (PE)—or who has previously manifested at least one symptom of such a disease or condition.

Hence, the invention relates to the use of idraparinux for the prevention of the recurrence of thromboembolism after discontinuation of the treatment with this drug.

In an embodiment, the invention relates to the use of idraparinux for the prevention of the recurrence of thromboembolism for an additional 6-month period after discontinuation of the treatment with this drug.

In another embodiment, the invention relates to the use of idraparinux for the prevention of the recurrence of thromboembolism after discontinuation of the treatment with this drug, wherein said treatment is initially administered for 6 months (i.e., the initial treatment period with idraparinux is 6 months).

In another embodiment, the invention relates to the use of idraparinux for the prevention of the recurrence of thromboembolism for an additional 6-month period after discontinuation of the treatment with this drug, wherein said treatment is initially administered for 6 months (i.e., the recurrence of thromboembolism is prevented for 6 months after the 6-month initial treatment period with idraparinux).

In another embodiment, the invention relates to a method for lowering the incidence of thromboembolism after discontinuation of a treatment with an anticoagulant drug, wherein said drug is idraparinux.

In another embodiment, the invention relates to a method for lowering the incidence of thromboembolism after discontinuation of a treatment with an anticoagulant drug, comprising the step of administering to a patient in need thereof a treatment with idraparinux. As described in the patent EP 1 471 924 B1, treatment with idraparinux is advantageously carried out with injections (e.g., subcutaneous injections) of a 2.5 mg dose once weekly.

In another embodiment, the method of the invention comprises the step of administering to a patient in need thereof a treatment with idraparinux for a 6-month period.

In another embodiment, the method of the invention lowers the incidence of thromboembolism in the 6-month period after discontinuation of the treatment with idraparinux.

In another embodiment, the invention relates to a pharmaceutical composition comprising idraparinux, useful for the prevention of the recurrence of thromboembolism after discontinuation of the treatment with this drug (idraparinux). Such a pharmaceutical composition advantageously comprises idraparinux, for instance at a weekly dose of 2.5 mg, as well as pharmaceutically acceptable and inert excipients. Such excipients are chosen among those known in the Art, according to the desired pharmaceutical formulation and mode of administration. An advantageous pharmaceutical composition according to the invention is an injectable formulation adapted to the subcutaneous route.

The term “thromboembolism” used in the instant invention is more particularly designating venous thromboembolism. Venous thromboembolism encompasses both deep venous thrombosis and pulmonary embolism.

The invention will be more clearly understood by reference to the following examples of the invention, which are included herewith for purposes of illustration only and are not intended to be limiting the invention.

A double-blind, placebo-controlled study evaluated patients who had completed 6 months of initial treatment for deep venous thrombosis (hereafter “DVT”) or pulmonary embolism (hereafter “PE”) and who were then randomly assigned to receive an additional 6 months of prophylaxis with idraparinux. The efficacy of this method of thromboprophylaxis was evaluated in these patients.

1) Patients

Eligible patients were those aged above or equal to 18 years, with confirmed, symptomatic DVT or PE and who had been treated for 6 months with a VKA (acenocoumarol or warfarin), either in previous van Gogh studies (see The van Gogh Investigators, N. Engl. J. Med., 2007, 357, 1094-104) or outside these studies, or with idraparinux (in van Gogh studies).

The diagnostic criteria for DVT were a calf trifurcation or a more proximal vein that were noncompressible on ultrasonography or an intraluminal filling defect on venography. For PE, the diagnostic criteria were an intraluminal filling defect in subsegmental or more proximal pulmonary arteries on spiral computed tomography (CT) or conventional pulmonary angiography, a high-probability finding on a ventilation-perfusion lung scan, or a nondiagnostic finding on a lung scan with objectively documented DVT (H. R. Büller et al., Ann. Intern. Med., 2004, 140, 867-73; The Matisse Investigators, N. Engl. J. Med., 2003, 349, 1695-702).

Patients were ineligible if they met one or more of the following criteria: an indication to continue anticoagulant therapy for the index DVT or PE beyond 6 months, other indications for prophylaxis with a VKA, pregnancy or breast-feeding, a creatinine clearance of less than 10 ml per minute, severe hepatic disease, bacterial endocarditis, active bleeding or a high risk of bleeding, uncontrolled hypertension (systolic blood pressure above 180 mm Hg or diastolic blood pressure above 110 mm Hg), or a life expectancy of less than 3 months.

Patients were randomized, with stratification according to study center and to the anticoagulant treatment received in the 6 months before randomization (idraparinux within the van Gogh studies or VKA within or outside the van Gogh studies). Randomization was to be performed not more than 1 hour before the administration of the first dose of the study drug.

2) Treatment

Patients received a once-weekly subcutaneous dose of 2.5 mg of idraparinux or placebo with an identical appearance for 6 months (26 injections). In patients who had received idraparinux before randomization, the first dose of idraparinux or placebo was given 6 to 8 days after the last injection of the drug. In patients who had received a VKA before randomization, the first dose was given when the international normalized ratio (INR) was 3.0 or less. For patients with a creatinine clearance of less than 30 ml/min., the weekly idraparinux dose was 1.5 mg, with a first dose of 2.5 mg in patients who had received a VKA before randomization.

3) Surveillance and Follow-Up

All patients were contacted after 1 week and at 3 and 6 months. All had an observation period of at least 3 months and up to 6 months after the study drug was stopped (regardless of whether the discontinuation was planned or premature). At each contact, symptoms and signs of recurrent VTE were investigated.

4) Outcome Assessment

All suspected outcome events were reviewed and classified by a central adjudication committee, whose members were unaware of study-group assignment.

The primary efficacy outcome was the incidence of symptomatic recurrence of VTE, defined as objectively documented recurrent PE, DVT or death attributed to PE. A diagnosis of recurrent VTE was accepted in the absence of an adequate objective test result if the investigator had treated the patient with therapeutic doses of heparin for more than 2 days or with a vena cava filter, thrombectomy or thrombolysis, or if recurrent VTE could not be ruled out.

The criteria for the objective diagnosis of recurrent PE were a new intraluminal filling defect on spiral CT or pulmonary angiography, a cutoff of a vessel of more than 2.5 mm in diameter on pulmonary angiography, a new perfusion defect of at least 75% of a segment with corresponding normal ventilation (high probability), a new non-high probability perfusion defect associated with DVT documented on ultrasonography or venography, or a new PE confirmed at autopsy.

The criteria for the objective diagnosis of a new DVT were a new, noncompressible venous segment or a substantial increase (4 mm or more) in the diameter of the thrombus during full compression in a previously abnormal segment on ultrasonography or a new intraluminal filling defect on venography.

The principal safety outcome was major bleeding. Bleeding was defined as major if it was clinically overt and associated with a fall in the hemoglobin level of 2 g per deciliter or more, if it led to the transfusion of two or more units of red cells, or if it was retroperitoneal or intracranial, occurred in a critical organ or contributed to death. All bleeding episodes that were clinically relevant but did not qualify as major bleeding (e.g. epistaxis that required an intervention or spontaneous macroscopic hematuria) constituted an additional safety outcome (The PERSIST Investigators, J. Thromb. Haemost., 2004, 2, 47-53; H. R. Büller et al., Ann. Intern. Med., 2004, 140, 867-73; The Matisse Investigators, N. Engl. J. Med., 2003, 349, 1695-702).

Deaths from all causes were also recorded. Death was classified as due to PE, bleeding, cancer or another established diagnosis. PE was considered to be the cause of death if the condition was objectively documented or if the cause of death was unexplained and PE could not be confidently ruled out.

5) Statistical Analysis

Analysis of the primary efficacy outcome, the incidence of symptomatic recurrent VTE at 6 months, was performed with the use of the Mantel-Haenszel test, stratified according to the previous treatment (idraparinux within the van Gogh studies or VKA within or outside the van Gogh studies). In addition, reductions in the adjusted risk and two-sided 95% confidence intervals were calculated with the use of the normal approximation of the log relative risk. Secondary planned efficacy analyses included a calculation of the cumulative incidence of recurrent VTE with the use of nonparametric Kaplan-Meier estimates.

Analysis of the primary safety outcome, the incidence of major bleeding at 6 months, was performed with the use of the Mantel-Haenszel test, stratified according to the previous treatment. The effect of initial, prerandomization treatment on efficacy and safety was also analyzed. All efficacy analyses were based on the total randomized population, whereas the safety analyses were based on the total randomized population that received at least one dose of a study drug.

6) Results

A total of 1215 patients underwent randomization in this study. Information on efficacy 6 months after randomization was complete in 99.8% of patients in the idraparinux group and in 99.7% of patients in the placebo group.

During the 6 months of randomly assigned treatment, 6 of 594 patients in the idraparinux group (1.0%) and 23 of 621 patients in the placebo group (3.7%) had symptomatic, objectively confirmed VTE (see Table 1). The reduction in the relative risk with idraparinux was 72.7% (95% confidence interval [CI], 33.5 to 88.8; P=0.002). Table 1 lists the types of recurrent episodes. Concerning the safety outcome, an increased risk of a major hemorrhage occurred in the idraparinux group (1.9%), compared to the placebo group (0%) (P<0.001).

TABLE 1
Clinical outcomes at month 6 after randomization, expressed
in number of patients/total number (%)

| | | | Odds Ratio | P |
|---|---|---|---|---|
| Outcome | Idraparinux | Placebo | (95% CI)¹ | Value |
| Recurrent | 6/594 (1.0%) | 23/621 (3.7%) | 0.27 (0.11-0.66) | 0.002 |
| VTE | | | | |
| Type of | | | | |
| recurrent | | | | |
| event²: | | | | |
| PE | | | | |
| fatal | 2 | 1 | | |
| non fatal | 1 | 11 | | |
| DVT only | 3 | 11 | | |
| Treatment | | | | |
| received | | | | |
| before | | | | |
| random- | | | | |
| ization: | | | | |
| Idraparinux | 3/255 (1.2%) | 2/268 (0.7%) | 1.58 (0.26-9.55) | 0.68 |
| VKA | 3/339 (0.9%) | 21/353 (5.9%) | 0.14 (0.04-0.48) | <0.001 |
| within study | 2/224 (0.9%) | 10/232 (4.3%) | 0.20 (0.04-0.92) | 0.02 |
| outside study | 1/115 (0.9%) | 11/121 (9.1%) | 0.09 (0.01-0.69) | 0.004 |

¹Odds ratio were adjusted according to treatment history.
²When patients had several recurrent events, they were counted in the category with the most severe outcome.

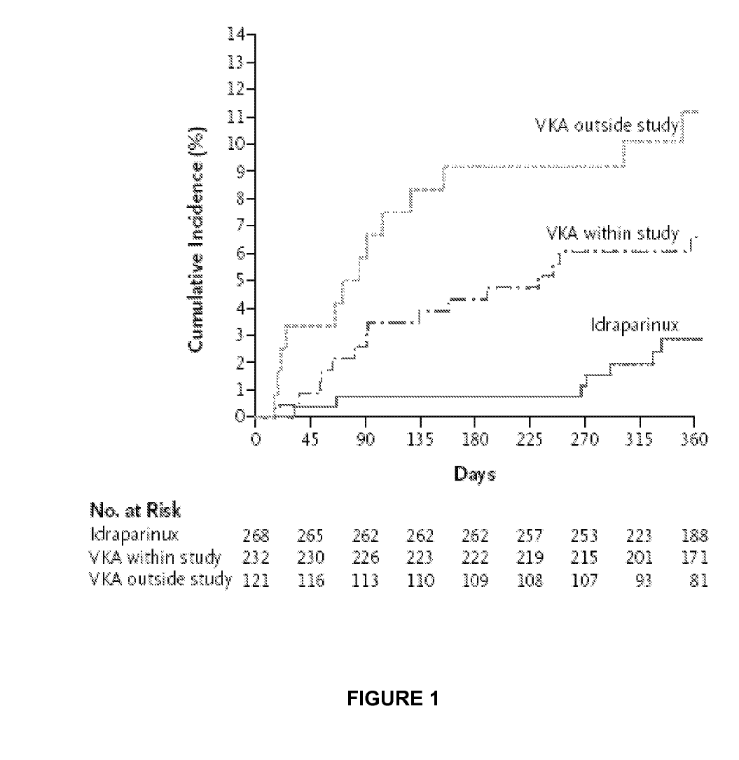

Additional observations have been made during a further 6-month observation period after the end of the additional 6-months of thromboprophylaxis as described above. During this period, 7 patients in the idraparinux group (1.2%) and 13 patients in the placebo group (2.1%) had recurrent VTE (P=0.21). The cumulative incidence of recurrent VTE 1 year after randomization, as estimated with the Kaplan-Meier method, was 1.7% in the idraparinux group and 5.9% in the placebo group (P=0.002). In the placebo group, the cumulative incidence of recurrent thromboembolism during the entire year after randomization was lowest in the group of 268 patients who had received idraparinux in the 6 months before randomization, as shown in FIG. 1, representing the Kaplan-Meier cumulative incidence of first confirmed symptomatic recurrent VTE in the placebo group, according to treatment received before randomization.

These results demonstrate that in patients with thromboembolism who had completed 6 months of anticoagulant therapy, a further 6 months of thromboprophylaxis with idraparinux reduced the frequency of recurrent thromboembolism to 1.0%, as compared with 3.7% in the placebo group.

Analyses of results within the placebo group lead to surprising conclusions. Indeed, among patients in the placebo group who had received VKA for 6 months before randomization, the frequency of recurrence was 5.9%, which is consistent with other studies (P. M. Ridker et al., N. Engl. J. Med., 2003, 348, 1425-34; S. Schulman et al., N. Engl. J. Med., 2003, 349, 1713-21). By contrast, in patients who first received idraparinux, the 6-month recurrence rate in the placebo group was less than 1%, without major bleedings. These results suggest a prolonged antithrombotic effect of idraparinux.

What is claimed is:

1. A method for the prevention of the recurrence of thromboembolism after discontinuation of an initial treatment for thromboembolism of a patient, the method comprising administering an initial treatment to a patient of idraparinux sufficient to treat or prevent a thromboembolism and then discontinuing such treatment.

2. The method of claim 1, wherein said prevention of the recurrence of thromboembolism is for an additional 6-month period after discontinuation of said initial treatment.

3. The method of claim 1, wherein the initial treatment with idraparinux is for 6 months.

4. The method of claim 2, wherein the initial treatment with idraparinux is for 6 months.

5. A method for lowering the incidence of thromboembolism after discontinuation of an initial treatment of a patient with an anticoagulant drug, the method comprising administering an initial treatment to a patient of idraparinux and then discontinuing such treatment.

6. The method of claim 5, wherein said initial treatment comprises the administration of idraparinux at a 2.5 mg dose once weekly.

7. The method of claim 5, wherein said initial treatment comprises the administration of idraparinux to the patient for a 6-month period.

8. The method of claim 5, wherein the incidence of thromboembolism is lowered for 6 months after discontinuation of said initial treatment with idraparinux.

9. The method of claim 6, wherein the incidence of thromboembolism is lowered for 6 months after discontinuation of said initial treatment with idraparinux.

10. The method of claim 7, wherein the incidence of thromboembolism is lowered for 6 months after discontinuation of said initial treatment with idraparinux.
